(19) United States
(12) Patent Application Publication
(10) Pub. No.: US 2004/0208924 A1
(43) Pub. Date: Oct. 21, 2004

(54) Pharmaceutical tablet having a high api content

(76) Inventors: Omar Leopold Sprockel, Bridgewater, NJ (US); Chiajen Lai, Kendall Park, NJ (US); Chenkou Wei, Princeton Junction, NJ (US); Sanjeev H. Kothari, Princeton, NJ (US); Robert P. Discordia, Pennington, NJ (US); Soojin Kim, West Orange, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/476,047

(22) PCT Filed: Apr. 23, 2002
(86) PCT No.: PCT/US02/12915

Related U.S. Application Data
(60) Provisional application No. 60/286,870, filed on Apr. 26, 2001.
(60) Provisional application No. 60/286,682, filed on Apr. 26, 2001.

(51) Int. Cl. A61K 38/04; A61K 31/44; A61K 31/16; A61K 31/40

(52) U.S. Cl. 424/464; 514/19; 514/357; 514/408; 514/616

(57) ABSTRACT

The invention is directed toward a tablet containing an unusually high percentage of an active ingredient in proportion to exipients.

### RELATED APPLICATIONS

This application claims priority benefit under Title 35 § 119(e) of U.S. provisional Application No. 60/286,682, filed Apr. 26, 2001, and U.S. provisional Application No. 60/286,870, filed Apr. 26, 2001. The contents of which are herein incorporated by reference.

### FIELD OF INVENTION

The present invention relates generally to a pharmaceutical tablet composition having an unusually high drug load.

### BACKGROUND OF THE INVENTION

Formulation of tablets used in the pharmaceutical industry usually involves the mixing of the active pharmaceutical ingredient (“API”) with excipient(s). Because the excipient tends to be the predominant portion of tablets, compaction typically entails excipient selection, enhancing the excipient's properties, or improving the process to mix or formulate the tablet. However, when a high API drug load is desired selection and/or manipulation of the excipient or process may not be enough to sufficiently compact the tablet. Furthermore, because of the high drug load, the mechanical properties (such as compactability) of the API predominate. The impact of insufficient compaction may lead to larger size tablets or the need for a patient to take more tablets then would be required if compaction were sufficient to obtain the desired drug load.

Currently, there are two general approaches to designing high drug load oral tablets containing API with low compactability (see Pharmaceutical Powder Compaction Technology, 1996, Ed. G. Alderborn and C. Nystrom, hereby incorporated by reference). The first approach is to add a pharmaceutically acceptable excipient(s) as a compaction aid. The second approach is to increase the compactability of the API through mechanical comminution. These two approaches are discussed in turn below.

In the first approach, the addition of excipient(s) to aid in compactibility does not address the deficiency in API compactability, but rather circumvents this shortcoming by the addition of excipients as a compaction aid. The addition of excipient(s) to a powder mixture does improve the performance of the powder mixture relative to that of the API; however, the addition of such compaction aids will lower the maximum API drug load per tablet, thereby increasing the size of the tablet per unit dose. This is commercially undesirable. In addition, these compaction aids are susceptible to a reduction in their compactability due to pharmaceutical processes, such as granulation. Hence, for optimal performance, these compaction aids should be matched with the API based on its mechanical characteristics.

In the second approach, API compactability is increased through the use of mechanical comminution (a.k.a, milling) which is an onerous process and can add significantly to drug product finishing costs. It is generally acknowledged that both particle size and particle shape (morphology) can have a dominant effect on material compactability. However, the effect of particle size on compaction can be positive or negative depending on the particular material studied (see, N. Kaneniwa, K. Imagawa, and J-C. Ichikawa, “The Effects of Particle Size and Crystal Hardness on the Compaction of Crystalline Drug Powders”, Powder Technology Bulletin Japan, 25 (6), 381 (1988), hereby incorporated by reference). In addition, the crystal morphology can be very critical to the amount of energy needed to bring the particles to full contact with each other therefore making a tablet with strong enough internal bonding strength. Further, comminution of API powder is a dusty and difficult operation, that is not friendly to large scale manufacturing. The level of increase in compactability with a reduction in API particle through mechanical means is unknown and may be insufficient to provide a high drug load tablet. Most importantly, a severe negative effect of mechanical comminution is the potential of increasing the amorphous content within the particles that could lead to serious stability problems.

Hence, there is often a need to produce strong, stable API containing tablets having high drug loads.

### SUMMARY OF THE INVENTION

The instant invention provides a pharmaceutical composition comprising at least 35% of an active ingredient. In one embodiment, the structure of the active ingredient is

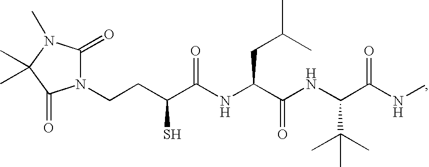

its enantiomers, diastereomers, pharmaceutically acceptable salts, hydrates, prodrugs and solvates thereof.

### DESCRIPTION OF DRAWINGS

FIG. 1 shows the nucleation and growth rate dependence on supersaturation.

FIG. 2 shows the process employed to increase the compactability of the API. It can be seen from FIG. 3 that on milling the API there was a gain in compactability after milling the API. However, milling the API also led to a reduction in the crystallinity of the API as seen from the X-ray diffraction patterns in FIG. 4. This amorphization through the milling process can lead to chemical instability of the API. It is also evident from FIG. 5 that particle size differences do not result in differences in degree of volume reduction. Hence, the differences in compactability are not related to the extent of volume reduction as the extent of volume reduction is independent of the particle size. This clearly illustrated that modification of the crystallization process parameters to achieve higher compactability of the API is the preferred choice.

FIGS. 6 through 15 are also provided to illustrate properties of the API.

FIG. 6 shows the particle size distribution of the API.

FIG. 7 shows data related to the compactability of the API.

FIG. 8 shows the compactability of the API with dry binders.

FIG. 9 shows the effect of particle size on the compressibility of the API.

FIG. 10 shows the effect of particle size on the extent of compaction of the API.

FIG. 11 shows the effect of seed amount and size during crystallization.

FIG. 12 shows the effect of seed size/amount on crystal structure.

FIG. 13 shows the performance of the API produced with Optimized Crystallization Conditions.

FIG. 14 shows the effect of speed on API tablet thickness.

FIG. 15 shows the effect of speed on API tablet breaking force.

FIG. 16 shows the compressibility of the API.

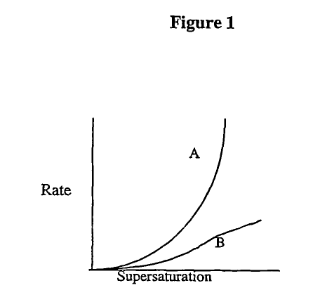

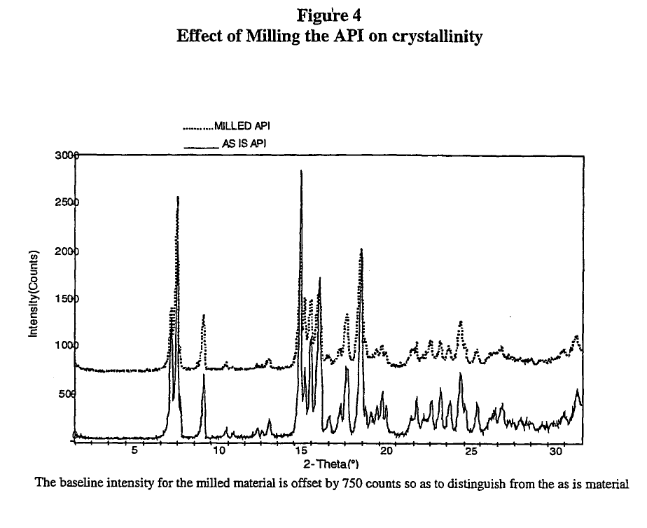

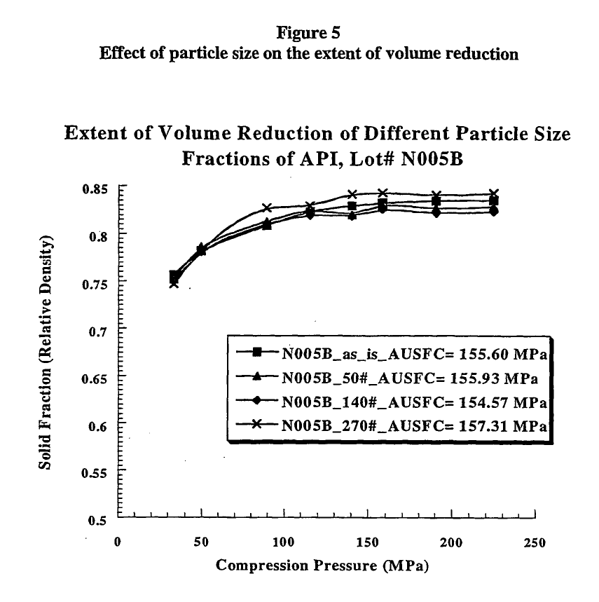

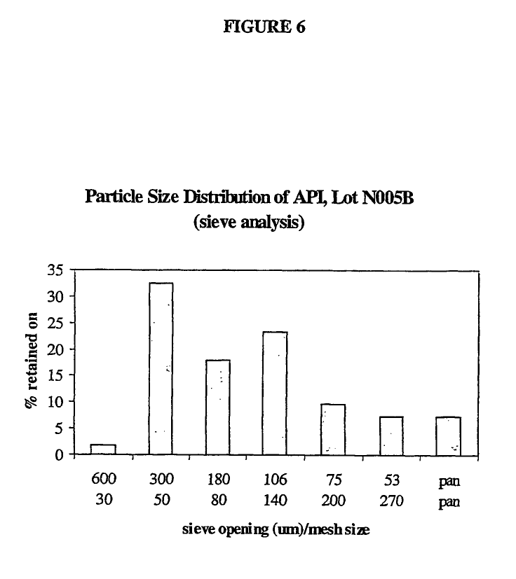

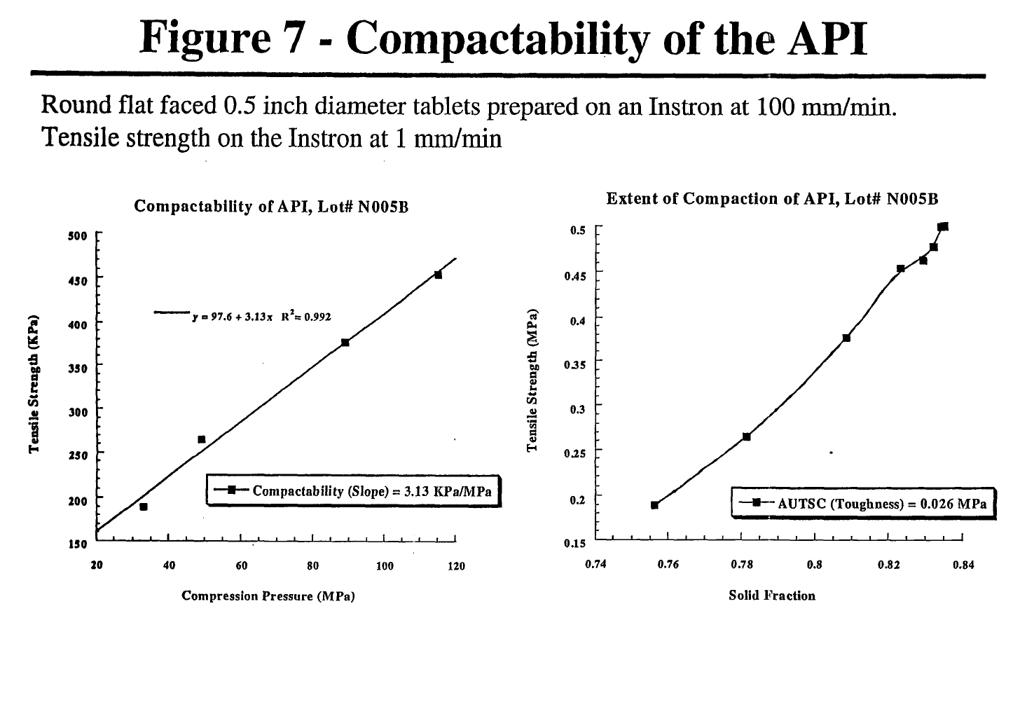

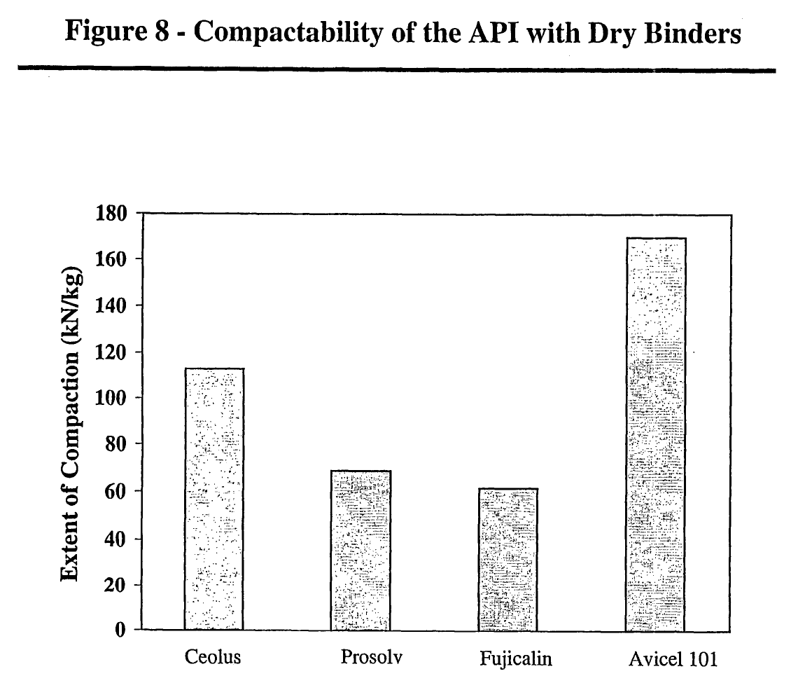

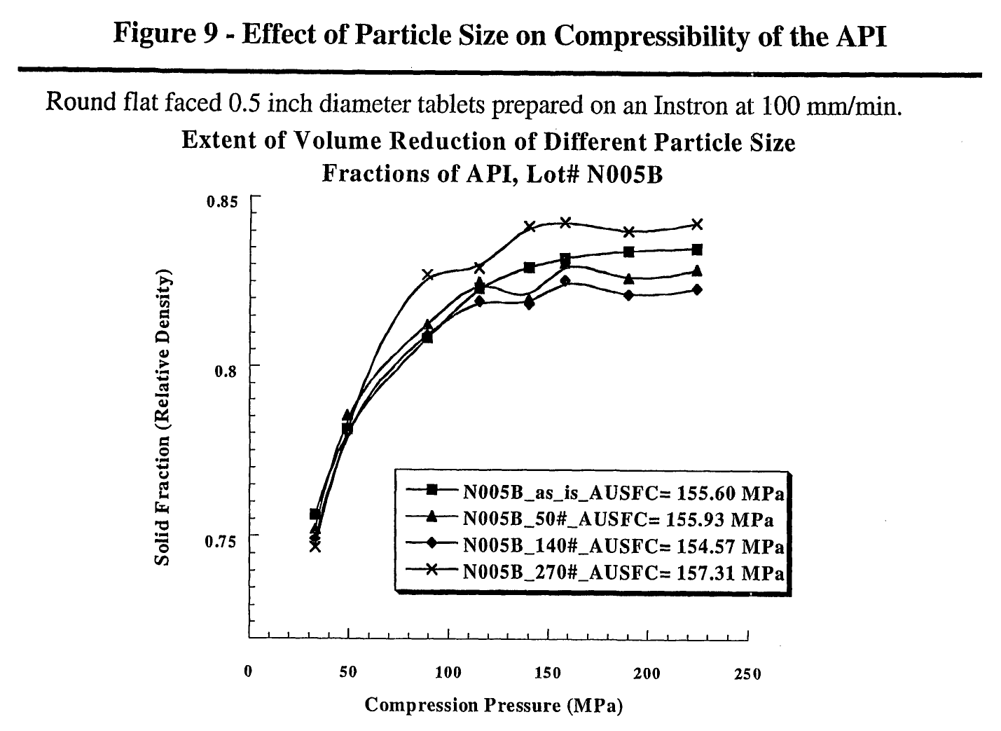

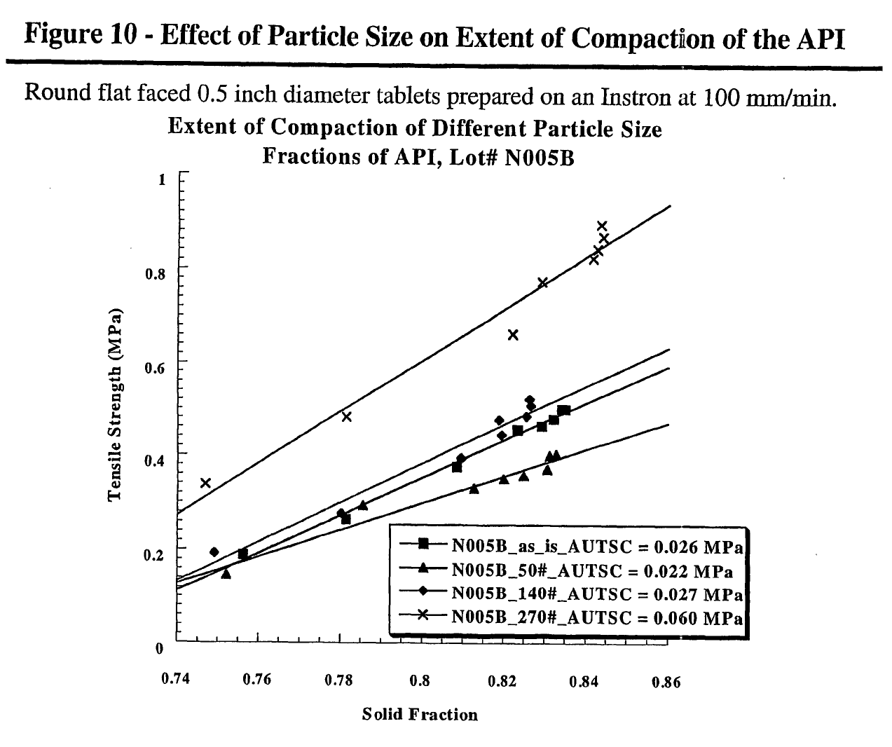

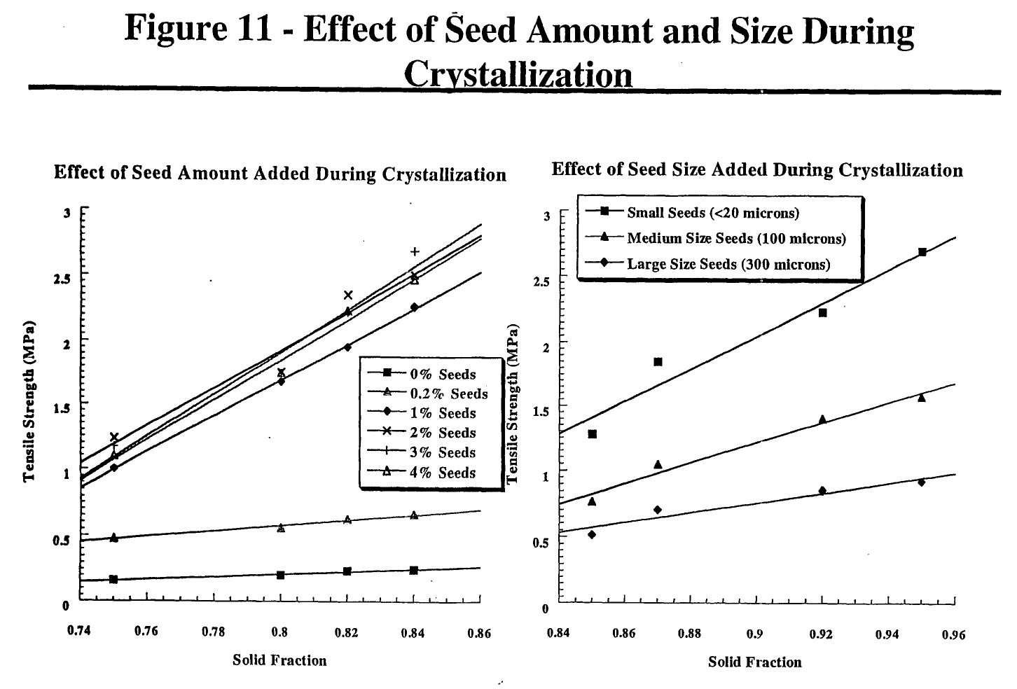

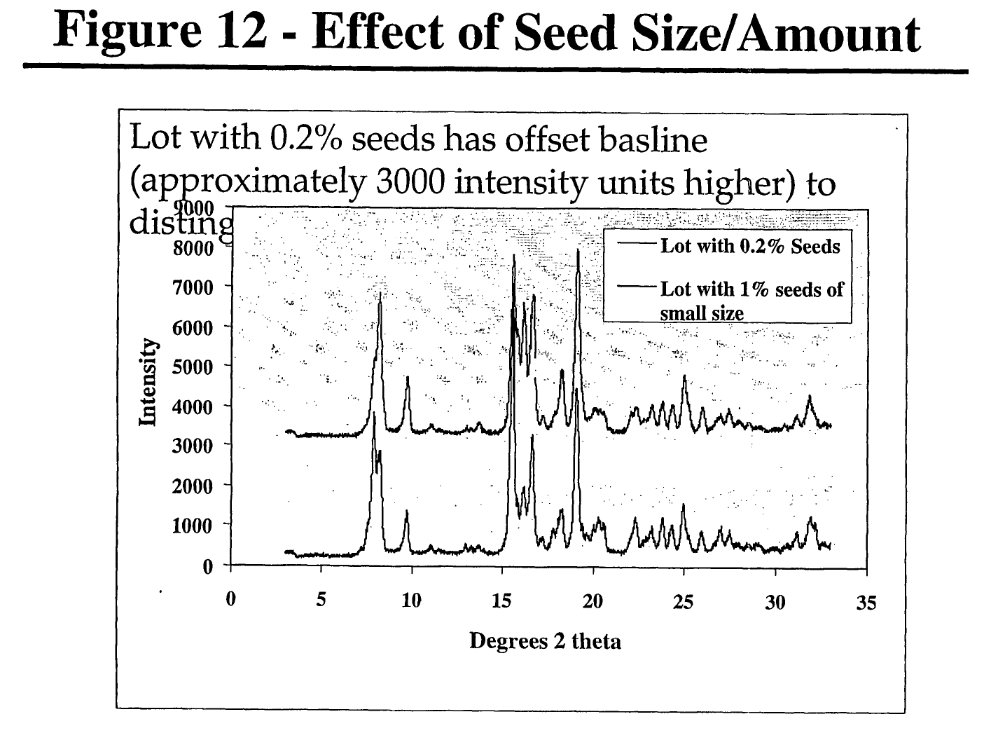

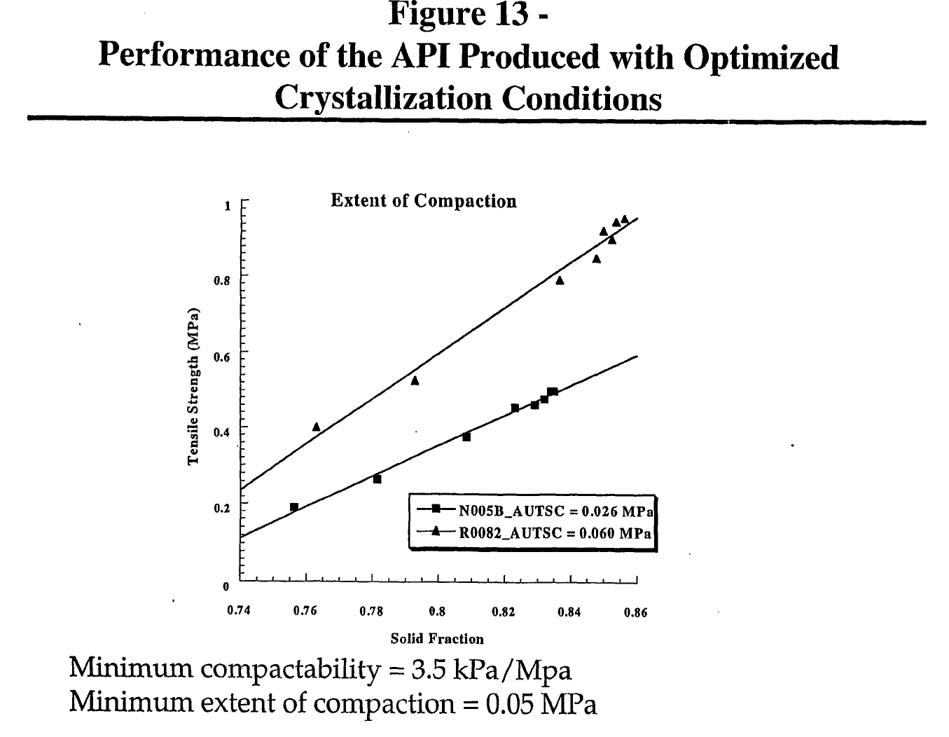

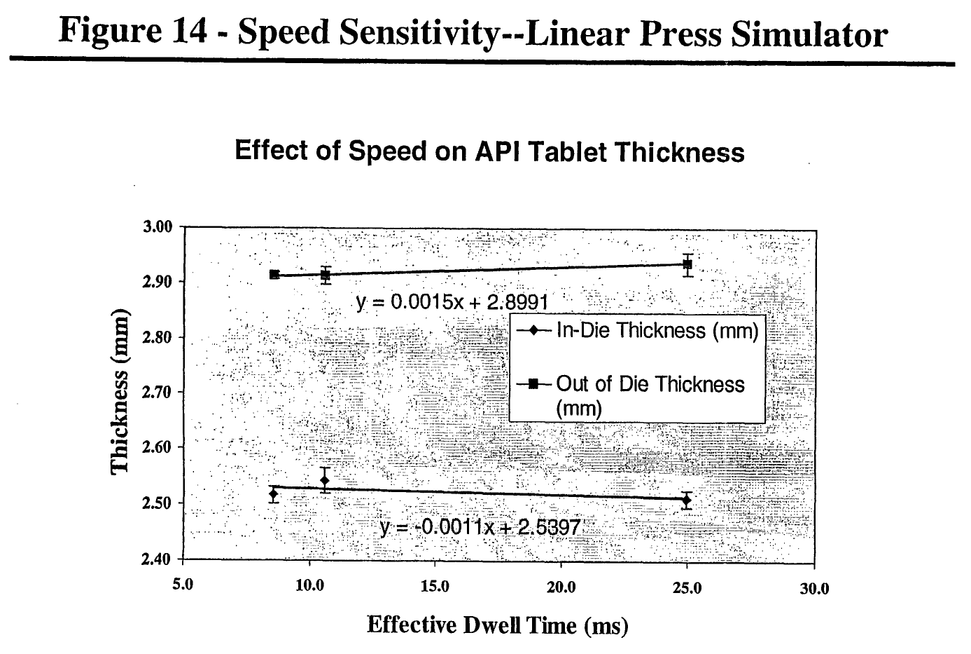

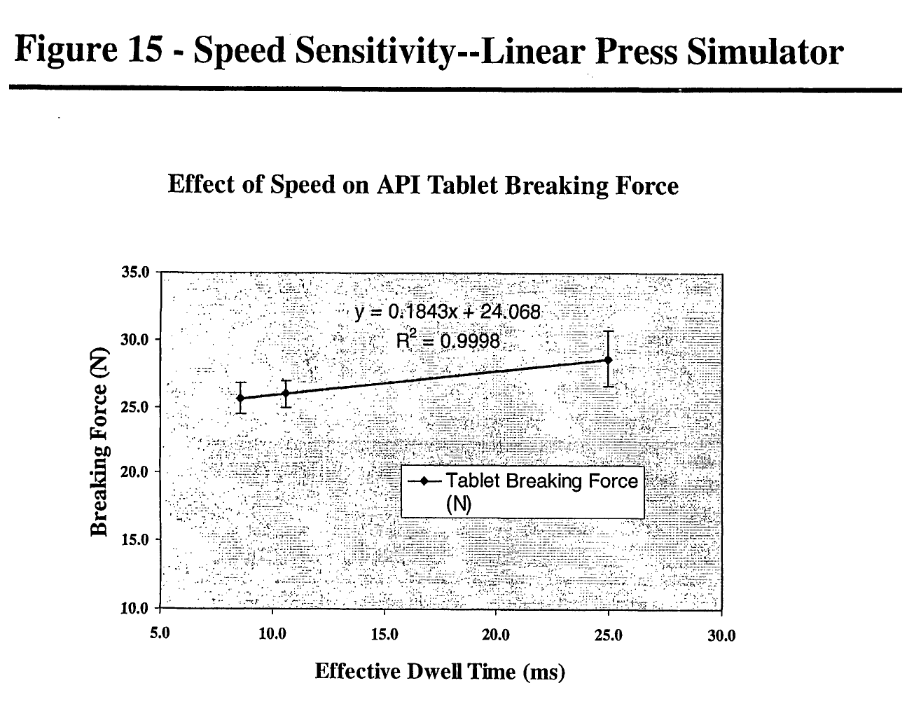

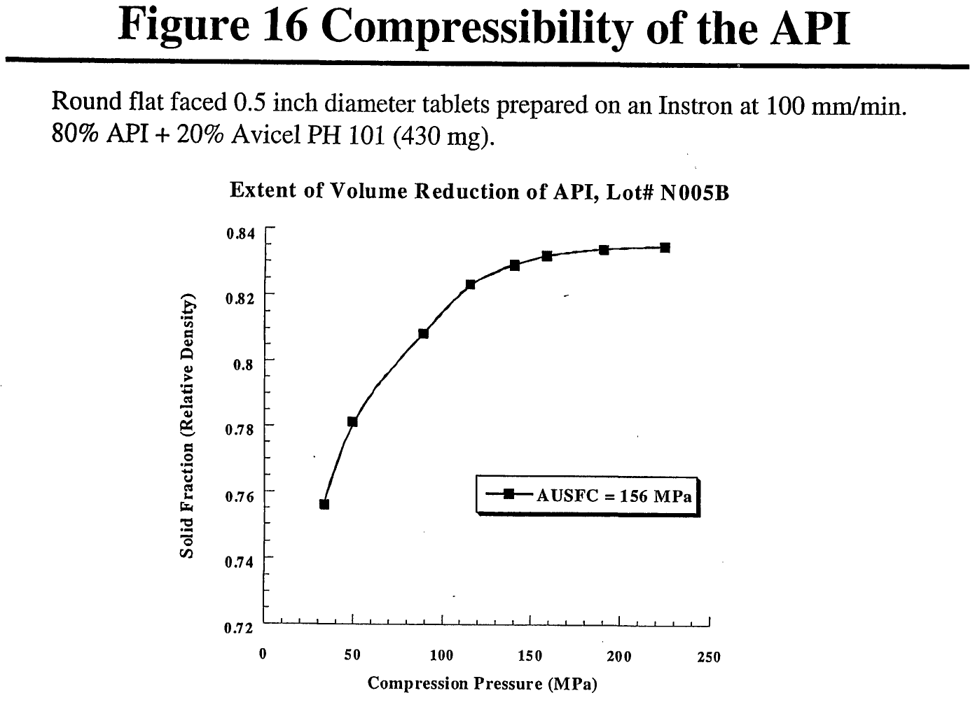

[Note: The API in FIGS. 1-16 is the compound of Example 1]

### DESCRIPTION OF THE INVENTION

The instant invention provides a pharmaceutical composition having an unusually high drug load. The drug load was increased by improving the compactability of an API by establishing a relationship between the crystallization parameters of the API and the compactability of the API. By establishing such a relationship it has been discovered that the improvement in API compactability could be achieved without the limitations of the conventional approaches described above.

Listed below are definitions and non-limiting descriptions of various concepts and techniques used to formulate, measure and evaluate various properties of APIs, excipients and tablets.

The term “AI” means active ingredient.

The term “API” means active pharmaceutical ingredient(s). “API” may also be referred to as AI “material”, “active agent” or “MMPI” (matrix metalloproteinase inhibitor).

The term “as is” (when referring to the “AI”, “API”, “active agent”, “MMPI” or “material”) means that the AI, API, active agent, MMPI or material has not gone through processing such as mechanical comminution or milling.

The term “excipient” means all ingredients other than the AI. Excipients used with the method of the instant invention shall include, but not limited to those described in the Handbook of Pharmaceutical Excipients, Second Edition, Ed. A. Wade and P. Weller, 1994, American Pharmaceutical Association, hereby incorporated by reference. In order to prepare a solid dosage form containing one or more active ingredients, it is often necessary that the material (which is to be compressed into the dosage form) possess certain physical characteristics which lend themselves to processing in such a manner. Among other things, the material to be compressed must be free-flowing, must be lubricated, and, importantly, must possess sufficient cohesiveness to insure that the solid dosage form remains intact after compression.

The phrase “high active ingredient content” means an amount of active ingredient in a tablet that is higher than would normally be attainable without using the novel process described herein.

The term “tablet” means a solid dosage form, which contains AI. Preferably it's a pharmaceutical tablet which contains API. The general process by which a tablet is formed should be evident to one skilled in the art; however, the following is a non-limiting description of the typical formation of a tablet and the equipmement, properties and materials which are used to form the tablets.

A tablet is formed by pressure being applied to the material to be tableted on a tablet press. A tablet press includes a lower punch which fits into a die from the bottom and a upper punch having a corresponding shape and dimension which enters the die cavity from the top after the tableting material fills the die cavity. The tablet is formed by pressure applied on the lower and upper punches. The ability of the material to flow freely into the die is important in order to insure that there is a uniform filling of the die and a continuous movement of the material from the source of the material, e.g. a feeder hopper. The lubricity of the material is crucial in the preparation of the solid dosage forms since the compressed material

must be readily ejected from the punch faces.

Since most drugs have none or only some of these properties, methods of tablet formulation have been developed in order to impart these desirable characteristics to the material(s) which is to be compressed into a solid dosage form. Typically, the material to be compressed into a solid dosage form includes one or more excipients which impart the free-flowing, lubrication, and cohesive properties to the drug(s) which is being formulated into a dosage form.

Lubricants are typically added to avoid the material(s) being tableted from sticking to the punches. Commonly used lubricants include magnesium stearate and calcium stearate. Such lubricants are commonly included in the final tableted product in amounts of less than 2% by weight.

In addition to lubricants, solid dosage forms often contain diluents. Diluents are frequently added in order to increase the bulk weight of the material to be tableted in order to make the tablet a practical size for compression. This is often necessary where the dose of the drug is relatively small. The choice of excipients used in dosage forms with a high drug load is essential to the mechanical performance of the formulation. For example, if the API is to be used in greater than 50% concentration may need to be balanced by use of ductile excipients. Conversely, if the API is ductile, one may want to use an excipient that would minimize the chances of the formulation being speed sensitive.

Another commonly used class of excipients in solid dosage forms are binders. Binders are agents which impart cohesive qualities to the powdered material(s). Commonly used binders include starch, and sugars such as sucrose, glucose, dextrose, lactose, povidone, methylcellulose, hydroxypropyl cellulose, and hydroxypropyl methylcellulose.

Disintegrants are often included in order to ensure that the ultimately prepared compressed solid dosage form has an acceptable disintegration rate in an environment of use (such as the gastrointestinal tract). Typical disintegrants include starch derivatives, salts of carboxymethyl cellulose, and crosslinked polymers of povidone.

There are three general methods of preparation of the materials to be included in the solid dosage form prior to compression: (1) dry granulation; (2) direct compression; and (3) wet granulation.

Dry granulation procedures may be utilized where one of the constituents, either the drug or the diluent, has sufficient cohesive properties to be tableted, The method includes mixing the ingredients, slugging or roller compacting the ingredients, dry screening, lubricating and finally compressing the ingredients.

In direct compression, the powdered material(s) to be included in the solid dosage form is compressed directly without modifying the physical nature of the material itself.

The wet granulation procedure includes mixing the powders to be incorporated into the dosage form in, e.g., a twin shell blender or double-cone blender and thereafter adding solutions of a binding agent to the mixed powders to obtain a granulation. Thereafter, the damp mass is screened, e.g., in a 6- or 8-mesh screen and then dried, e.g., via tray drying, the use of a fluid-bed dryer, spray-dryer, radio-frequency dryer, microwave, vacuum, or infra-red dryer. The dried granulation is dry screened, lubricated and finally compressed.

The use of direct compression is typically limited to those situations where the drug or active ingredient has a requisite crystalline structure and physical characteristics required for formation of a pharmaceutically acceptable tablet. On the other hand, it is well known in the art to include one or more excipients which make the direct compression method applicable to drugs or active ingredients which do not possess the requisite physical properties. For solid dosage forms wherein the drug itself is to be administered in a relatively high dose (e.g., the drug itself comprises a substantial portion of the total tablet weight), it is necessary that the drug(s) itself have sufficient physical characteristics (e.g., cohesiveness) for the ingredients to be directly compressed.

A rational selection of manufacturing process has to be made based on the deformation mechanism of the active ingredient. For example, avoid dry granulation with very brittle materials, while choosing wet granulation in order to overcome elasticity issues.

Typically, however, excipients are added to the formulation which impart good flow and compression characteristics to the material as a whole which is to be compressed. Such properties are typically imparted to these excipients via a pre-processing step such as wet granulation, slugging or roller compaction, spray drying, spheronization, or crystallization. Useful direct compression excipients include processed forms of cellulose, sugars, and dicalcium phosphate dihydrate, among others.

A processed cellulose, microcrystalline cellulose, has been utilized extensively in the pharmaceutical industry as a direct compression vehicle for solid dosage forms. Microcrystalline cellulose is commercially available under the tradename EMCOCEL™ from Edward Mendell Co., Inc. and as Avicel™ from FMC Corp. Compared to other directly compressible excipients, microcrystalline cellulose is generally considered to exhibit superior compressibility and disintegration properties.

The preferred size of a commercially viable tablet is constrained on the low side (approximately 100 mg) by a patients ability to handle it, and on the high side (approximately 800 mg) by the ease of swallowing. These weights assume a formula of average density (0.3 g/mL to 0.6 g/mL). The desired tablet weight range is 200 mg to 400 mg. The preferred formulation would possess the desired properties of good flow and good compactability, but at the same time requiring the least amount of excipients to overcome any deficiency in the API physical properties. Hence, it is advantageous to have the API possess as much of the desired qualities as possible.

Generally, to form an AI containing tablet, a given weight of powder bed (constituted of the AI or a mixture thereof with excipient(s)) is subjected to compression pressure in a confined space, as in a die between the upper and lower punch, it undergoes volume reduction leading to consolidation, thereby forming a tablet. The change in volume that occurs due to the applied pressure can be measured from the dimensions of the resulting tablet. The extent of volume change over the pressure range applied represents the extent of compression or volume reduction that the material undergoes. Similarly the slope or response of volume change with respect to pressure represents the compressibility of the powder. Consolidation occurs due to fresh new surfaces generated through the volume reduction process (either a plastic deformation or brittle fracture) that come in close contact at distances where interparticulate bonds become active. These bonds could be either intermolecular forces or weak dispersion forces depending on the juxtaposition of the contact points and the chemical environment existing around them. The consolidated powder bed, now a tablet, has a strength of its own that allows it to resist failure or further deformation when subjected to mechanical stress. The strength of the tablet can be conveniently measured in terms of a tensile test. In a “tensile test”, the tablet is subjected to stress in a direction perpendicular to its plane having the longest width/diameter. The strength determined from this test is known as the “tensile strength” of the tablet.

API powders generally show greater degree of consolidation with increasing compression pressure. However, it is virtually impossible to produce a compact that has no air in it or, in other words, is a 100% solid body. With increasing consolidation, there is in general, an increase in the tensile strength of the compact produced. The measure of increase in strength with increasing compression pressure (slope) is used as a measure of the ability of the material to respond to compression pressure or the “compactability”. The extent of compaction can also be monitored by measuring the area under the curve of such a profile as described in the preceding sentence.

The instant invention was produced by engineering those properties that enhance its compactability into the API material to be compacted. There are several crystallization parameters which can be systematically studied for their effect on material compactability. Examples of such crystallization parameters include, but are not limited to, sonication, seed size, seed amount, volume of antisolvent, crystallization temperature, cooling profile, rate of agitation, as well as other parameters known to those skilled in the art. Generally, the crystallization process involves both nucleation and growth. Their empirical dependence on supersaturation is shown in FIG. 1 which is a schematic representation of the nucleation (homogeneous, unseeded; Curve A) and growth rate (Curve B) dependence on supersaturation. One way to manipulate the crystallization process is to control the degree of supersaturation For example, if large particle size is desirable, one can reduce supersaturation and therefore decrease the rate of nucleation and let the material in solution to crystallize/deposit upon existing crystals which serves as nucleates. On the other hand, if small particle size is desired, higher supersaturation usually force an increase in nucleation rate and consequently material in solution would prefer to initiate a nucleate and start a new crystal entity. The shape of the crystals (morphology), or the crystallization habit of the crystals, may or may not be changed by this modification depending on the material of interest. Through the manipulation of the supersaturation, it is possible to control the compactability of the end product AI.

Another way to modify the crystallization process is to enhance nucleation by introducing more seeds or to preclude nucleation by using no seeds at all and shift the balance between nucleation and growth for a specific degree of supersaturation. This approach is especially useful for materials with an extremely slow or fast nucleation rate.

For example, in a crystallization system where nucleation is slow and if only limited amount of seeds are present, supersaturation tends to drive the material in solution to grow upon the seeds instead of initiating new crystals. The results will be larger crystals upon the completion of the crystallization. Although there are other factors (e.g. the selection of different solvents) which might affect the morphology of the particles and therefore impact their performance, the application of excessive seeding definitely provides a powerful tool to control the particle size and accordingly the compactability of the product.

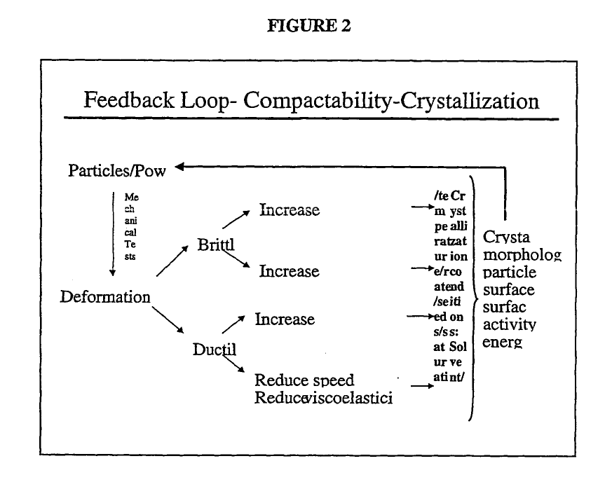

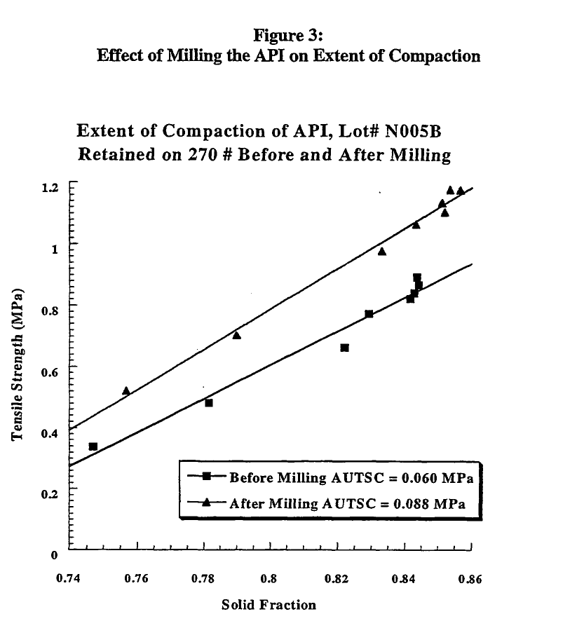

FIG. 2 is provided as a non-limiting aid to help understand the overall process of increasing the compactability of the API. As such, FIG. 2 shows a feedback loop wherein the AI particles, or blends of AI and excipient(s), are evaluated for their deformation mechanism using mechanical tests such as the tablet indices procedure described herein. Further, other techniques such as the compressibility and compactability experiments described herein are used to help identify whether the AI is predominantly brittle or ductile under compression stress. If the AI is found to be brittle, the crystallization process is modified using the approaches described herein so as to achieve maximum compressibility and compactability by altering the crystal morphology/size/shape/surface area/surface energy. If the AI is determined to be ductile but exhibits low tensile strengths then the route of altering the crystallization process is taken to achieve maximum compactability. However, if tensile strength is not the issue but viscoelasticity is, then the crystallization approach can look at how the crystals can be made harder (e.g. high temperature treatment, etc.) The modified crystals and resulting powders are then re-evaluated for their mechanical properties through the feedback loop until the desired properties are attained.

The invention provides a tablet comprising a high active ingredient content wherein said active ingredient is of the general formula (I):

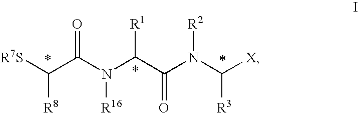

where R¹ is C₁₋₇ alkyl, C₂₋₆ alkenyl, C₁₋₆ alkyl-aryl, aryl, C₁₋₆ alkyl-heteroaryl, heteroaryl or

C₁₋₆ alkyl-AR⁹ group where A is O, NR⁹ or S(O)ₘ where m=0-2, and R⁹ is H, C₁₋₄ alkyl, aryl, heteroaryl, C₁₋₄ alkyl-aryl or C₁₋₄ alkyl-heteroaryl; if A=NR⁹ the groups R⁹ may be the same or different,

R² is hydrogen or a C₁₋₆ alkyl group;

R³ is a R⁶ group where Alk is a C₁₋₆ alkyl or C₂₋₆ alkenyl group and n is zero or 1;

X is heteroaryl or a group CONR⁴R⁵ where R⁴ is hydrogen or an C₁₋₆ alkyl, aryl, heteroaryl, C₁₋₆ alkyl-heteroaryl, cyclo(C₃₋₆)alkyl, C₁₋₆ alkyl-cyclo(C₃₋₆)alkyl, heterocyclo(C₄₋₆)alkyl or C₁₋₆ alkyl-heterocyclo(C₄₋₆)alkyl group and R⁵ is hydrogen or C₁₋₆ alkyl; NR⁴R⁵ may also form a ring;

R⁷ is hydrogen or the group R¹⁰CO where R¹⁰ is C₁₋₄ alkyl, (C₁₋₄ alkyl)aryl, (C₁₋₆ alkyl)heteroaryl, cyclo(C₃₋₆)alkyl, cyclo(C₃₋₆)alkyl-C₁₋₄ alkyl, C₂₋₆ alkenyl, C₂₋₆ alkenylaryl, aryl or heteroaryl;

R⁸ and R¹⁶ are the same or different and are each C₁₋₄ alkyl R¹¹, R¹⁶ may also be H;

R⁶ represents AR⁹ or cyclo(C₃₋₆)alkyl, cyclo(C₃₋₆)alkenyl, C₁₋₆ alkyl, C₁₋₆ alkoxyaryl, benzyloxyaryl, aryl, heteroaryl, (C₁₋₃ alkyl)heteroaryl, (C₁₋₃ alkyl)aryl, C₁₋₆ alkyl-COOR⁹, C₁₋₆ alkyl-NHR¹⁰, CONHR¹⁰, NHCO₂R¹⁰, NHSO₂R¹⁰, NHCOR¹⁰, amidine or guanidine;

R¹¹ is COR¹³, NHCOR¹³ or any of the groups

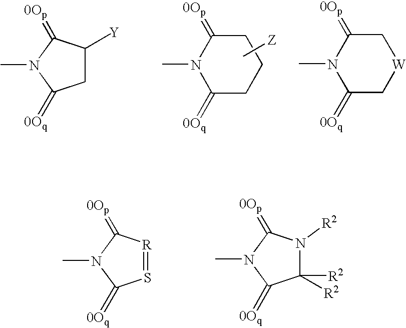

where p and q are each 0 or 1 and are the same or different but when p=q=1, Y cannot be H;

R and S are each CH or N and are the same or different;

W is O, S(O)ₘ where m=0, 1 or 2 or NR¹²;

Y and Z are each H or C₀₋₄ alkylR¹⁴ wherein R¹⁴ is NHR², N(R²)₂ (where each R² may be the same or different), COOR², CONHR², NHCO²R² (where R² is not H), NHSO₂R² (where R² is not H) or NHCOR²; Z may be attached to any position on the ring;

R¹² is hydrogen, C₁₋₄ alkyl, COR⁹, CO₂R⁹ (where R⁹ is not H), CONHR⁹, or SO₂R⁹ (where R⁹ is not H);

R¹³ is (C₁₋₄ alkyl)R¹⁵;

R¹⁵ is N(R²)₂ (where each R⁹ may be the same or different), CO₂R⁹, CONHR⁹, CON(R⁹)₂ (where each R₉ may be the same or different) or SO₂R⁹ (where R⁹ is not H), phthalimido or the groups

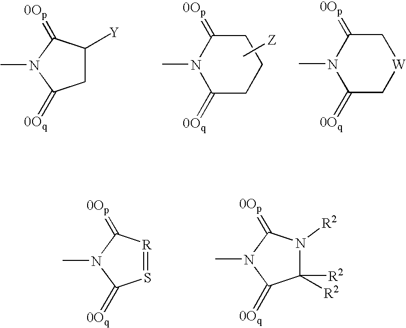

as defined above;

and the salts, solvates and hydrates thereof.

Typically, the high active ingredient content is greater than 35% of the composition. Preferably, the high active ingredient content is greater than 50%; more preferrably it's greater 60%; even more preferrably it's greater than 70%; still more preferrably it's greater than 80%; most preferrably it's greater than 90%.

In a preferred embodiment, the AI is a compound of formula I, wherein X is CONR⁴R⁵; R⁴ is H, alkyl or aryl; R⁶ is not amidine or guanidine; R¹¹ is not NHCOR¹³ or the last of the given groups; R¹⁵ is not N(R²)₂ or the last of the given groups; and R¹⁶ is H.

In a preferred embodiment, the AI is a compound of formula I selected from the group consisting of

[(2S)-Sulfanyl-5-[(N,N-dimethylamino)acetyl]aminopentanoyl-L-leucyl-L-tert-leucine N-methylamide; and

[(2S)-Sulfanyl-5-[(N-methylamino)acetyl]aminopentnoyl-L-leucyl-L-tert-leucine N-methylamide.

In a preferred embodiment, the AI is a compound of formula I selected from the group consisting of

[(2S)-Acetylthio)-4(1,5,5-trimethylhydantoinyl)butanoyl]-L-Leucyl-L-tert-leucine N-methylamide;

[(2S)-Acetylthio)-4(1,5,5-trimethylhydantoinyl)butanoyl]-L-(S-methyl)cysteinyl-L-tert-leucine N-methylamide;

[(2S)-Acetylthio)-4(1,5,5-trimethylhydantoinyl)butanoyl]-L-norvalinyl-L-tert-leucine N-methylamide;

N-[2-Sulfanyl-4-(1,5,5-trimethylhydantoinyl)butanoyl]-L-leucyl-L-tert-leucine N-methylamide;

N-[2-Sulfanyl-4-(1,5,5-trimethylhydantoinyl)butanoyl]-L-(S-methyl)cysteinyl-L-tert-leucine N-methylamide; and

N-[2-Sulfanyl-4-(1,5,5-trimethylhydantoinyl)butanoyl]-L-norvalinyl-L-tert-leucine N-methylamide.

In a preferred embodiment, the AI is a compound of formula I in the form of a single enantiomer or diastereomer, or a mixture of such isomers.

In a preferred embodiment, the AI is a compound of formula I, wherein the ring formed from NR⁴R⁵ is pyrrolidino, piperidino or morpholino.

In a preferred embodiment, the AI is a pharmaceutical composition comprising a compound of formula I, and a pharmaceutically-acceptable diluent or carrier.

In a preferred embodiment, the tablet is a pharmaceutical composition as described above, wherein said pharmaceutical composition is formulated to be administered to a human or animal by a route selected from the group consisting of oral administration, topical administration, parenteral administration, inhalation administration and rectal administration.

In a preferred embodiment, the tablet is a pharmaceutical composition used for the treatment in a human or animal of a condition associated with matrix metalloproteinases or that is mediated by TNF.α, or L-selectin sheddase, wherein the tablet comprises a therapeutically effective amount of a compound of the formula I.

In a preferred embodiment, the tablet is a pharmaceutical composition for the treatement of conditions selected from the group consisting of cancer, inflammation and inflammatory diseases, tissue degeneration, periodontal disease, ophthalmological disease, dermatological disorders, fever, cardiovascular effects, hemorrhage, coagulation and acute phase response, cachexia and anorexia, acute infection, HIV infection, shock states, graft versus host reactions, autoimmune disease, reperfusion injury, meningitis and

migraine.

In a preferred embodiment, the tablet is a pharmaceutical composition for the treatement of conditions selected from the group consisting of tumour growth, angiogenesis, tumour invasion and spread, metastases, malignant ascites and malignant pleural effusion.

In a preferred embodiment, the tablet is a pharmaceutical composition for the treatement of conditions selected from the group consisting of rheumatoid arthritis, osteoporosis, asthma, multiple sclerosis, neurodegeneration, Alzheimer's atherosclerosis, stroke, vasculitis, Crohn's disease and ulcerative colitis.

In a preferred embodiment, the tablet is a pharmaceutical composition for the treatement of conditions selected from the group consisting of corneal ulceration, retinopathy and surgical wound healing.

In a preferred embodiment, the tablet is a pharmaceutical composition for the treatement of conditions selected from the group consisting of psoriasis, atopic dermatitis, chronic ulcers and epidermolysis

bullosa.

In a preferred embodiment, the tablet is a pharmaceutical composition for the treatment of conditions selected from the group consisting of periodontitis and gingivitis.

In a preferred embodiment, the tablet is a pharmaceutical composition for the treatement of conditions selected from the group consisting of rhinitis, allergic conjunctivitis, eczema and anaphylaxis.

In a preferred embodiment, the tablet is a pharmaceutical composition for the treatment of conditions selected from the group consisting of restenosis, congestive heart failure, endometriosis, atherosclerosis and endosclerosis.

In a preferred embodiment, the tablet is a pharmaceutical composition for the treatement of osteoarthritis.

In a preferred embodiment, the instant invention provides a pharmaceutical composition comprising at least 35% of an active ingredient having the structure

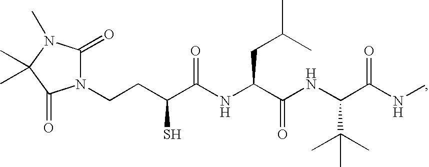

its enantiomers, diastereomers, pharmaceutically acceptable salts, hydrates, prodrugs and solvates thereof. This compound has been demostrated to be an effective matrix metalloproteinase inhibitor (MMPI) as well as a tumor necrosis factor α(TNFα). Examples of the matrix metalloproteinases include collagenase and stromelysin (see PCT International application publication WO 97/12902 and U.S. Pat. No. 5,981,490, both of which are herein incorporated by reference). The invention may further comprise at least one excipient.

In a preferred embodiment, active ingredient comprises at least 50% of the composition. In another preferred embodiment, the active ingredient comprises at least 60% of the composition. In another preferred embodiment, the active ingredient comprises at least 70% of the composition. In still yet another preferred embodiment, the active ingredient comprises at least 80% of the composition. In another embodiment the active ingredient comprises at least 90% of the composition.

In a preferred embodiment, the excipient is selected from the group consisting of microcrystalline cellulose, sodium starch glycolate, silicon dioxide and magnesium stearate. In a further preferred embodiment, the active ingredient is about 50 to 90% of the composition.

All the compositions described above may further comprising microcrystalline cellulose, sodium starch glycolate, silicon dioxide and magnesium stearate.

In a further preferred embodiment, the active ingredient is about 70 to 90% of the composition.

In still yet another preferred embodiment said active ingredient is about 80% of the composition; said microcrystalline cellulose is about 13% of the composition; said sodium starch glycolate is about 5% of the composition; said silicon dioxide is about 1.25%; and said magnesium stearate is about 0.75%.

In a preferred embodiment, the pharmaceutical composition is in a solid dosage form. In another preferred embodiment, said pharmaceutical composition is a tablet. In yet another preferred embodiment, the pharmaceutical composition is an oral tablet.

In a preferred embodiment, the composition further comprises at least one excipient having desirable mechanical properties. An excipient so selected should have a high compressibility, a high compactability, a high bonding index, and a low brittle fracture index. The methodology to determine these properties is described herein. Preferred excipients include microcrystalline cellulose, sodium starch glycolate, silicon dioxide and magnesium stearate. Other preferred excipients include diluents: lactose, maltodextrin, Mannitol, sorbitol, sucrose, calcium phosphate; disintegrants: Croscarmellose sodium, crospovidone, pregelatinized starch; lubricants: stearic acid, sodium stearate, calcium stearate, sodium stearyl fumarate; and glidant, talc.

### EXAMPLE 1

### Producing a High API Load (80%) Oral Tablet Dosage Form

The API used in the instant invention has the structure

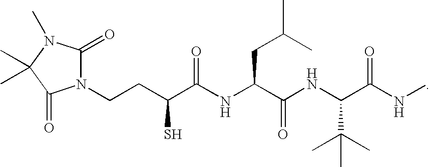

This API and the procedure to make this API are fully described in U.S. Pat. No. 5,981,490, WO 97/12902 and co-pending U.S. patent application Ser. No. 09/961,932 filed Sep. 24, 2001, all of which are hereby incorporated by reference. This API is also referred to herein by its Chemical Abstracts Systematic Name, N-[(2S)-2-Mercapto-1-oxo-4-(3,4,4-trimethyl-2,5-dioxo-1-imidazolidinyl)butyl]-L-leucyl-N,3-dimethyl-L-valinamide

(Chemical Abstracts Systematic Number: 259188-38-0).

Due to the unique structure of the API material at least four different groups of crystal structures were observed (forms 4, 5, 6, 7) and analyzed by single crystal x-ray. Orthorhombic Form 5 and monoclinic Form 7 (both solvates) were found to have similar molecular conformations containing solvent cavities which may accommodate CHCl₃, IPA, acetone, and MEK, etc. Orthorhombic Form 6 consisted of a group of isostructural (1:1) solvates which accommodates solvents such as EtOAc, acetone and MEK. Out of the four crystal structures the Form 4 (a triclinic de-solvated form) was the only one which did not transform/decompose to other crystalline structures in the solid state and was thus selected for development. An exhaustive study of API crystallization on the feasibility of various solvents, control of polymorphs, and robustness of process concluded that the selected form could be consistently produced and kept stable in iPrOAc (or BuOAc)/Heptane (or Cyclohexane), following which a reproducible crystallization procedure in the iPrOAc/heptane solvent system was developed and implemented. This procedure, associated with the aminolysis of penultimate compound (Chemical Abstracts Systematic Name, (αS)-α-(Benzoylthio)-3,4,4-trimethyl-2,5-dioxo-1-imidazolidinebutanoyl-L-leucyl-N,3-dimethyl-L-valinamide), is successful in purging undesirable side products/impurities such as α,α′-Dithiobis[N-[1-[[[2,2-dimethyl-1-[(methylamino)carbonyl]propyl]amino]-carbonyl]-3-methylbutyl]-3,4,4-trimethyl-2,5-dioxo-1-imidazolidinebutanamide] which is the S,S′-dimer of the API. The crystallization procedure is further described in Table 1.

TABLE 1
Preliminary crystallization procedure of the API
in iPrOAc/Heptane solvent system

| | |
|---|---|
| 1 | Post-aminolysis reaction mixture which contains impurities and 10 g |
| | of the API(N-[(2S)-2-Mercapto-1-oxo-4-(3,4,4-trimethyl-2,5-dioxo-1- |
| | imidazolidinyl)butyl]-L-leucyl-N,3-dimethyl-L-valinamide) added |
| | with 30 mL iPrOAc (1 g/3 mL) is dissolved at 75-80° C. (the final |
| | volume of the solution is 37-38 mL) |
| 2 | The solution is held at a temperature of 75-80° C. |
| 3 | Charge ˜20 mL heptane while maintaining the temperature of the |
| | solution at 75-80° C. Up to this point there is no solid present in the |
| | crystallization solution. |
| 4 | Seed the crystallization solution with ˜20 mg (0.2% wt.) of the API |
| 5 | Hold the solution at 75-80° C. for 1-2 hours |
| 6 | Charge another ˜20 mL heptane while maintaining the temperature of |
| | the solution at 75-80° C. A slow rate of heptane addition is |
| | recommended to avoid localized nucleation. |
| 7 | Hold the slurry at 75-80° C. for another 1-2 hours |
| 8 | Cool the solution at a linear steady rate from 75-80° C. to ambient |
| | temperature over 4 hours and hold for 1-2 hours |
| 9 | Isolated the product by filtration on a Buchner funnel and Whatman |
| | #1 filter paper |
| 10 | Dry the solid cake under vacuum at no more than 55° C. until there is |
| | no further weight change. |

The following illustrates how compactability of the API (N-[(2S)-2-Mercapto-1-oxo-4-(3,4,4-trimethyl-2,5-dioxo-1-imidazolidinyl)butyl]-L-leucyl-N,3-dimethyl-L-valinamide) was improved through the control of crystallization parameters.

The crystallization parameters and seeding conditions (using “as is” API at 0.1-0.2%) described in the procedure outlined in Table 1 was adopted as a starting point for modifications. By changing the ratio of solvent/antisolvent (isopropyl acetate/heptane) in step 3 (Table 1) from 1.67 to 1.0 and varying the pot temperature from 80 to 50° C., the degree of supersaturation was increased by a factor of 5 (from about 3.5 to about 17.5). The materials made from these conditions are generally agglomerates formed by a cluster of primary crystals plus the conjunction material which glue these crystals together.

At low supersaturation, large agglomerates (500-1000 μM) with large primary crystals (also large) were obtained. At high supersaturation the procedure generates small agglomerates (200-300 μm) with smaller primary crystals. This is consistent with other crystallization systems, in which nucleation is rate limiting, where high supersaturation favors the formation of agglomerates and mild supersaturation results in elementary crystals. Generally, these agglomerated materials compact quite poorly and create difficulties for large scale, high speed tablet manufacture. In addition, the agglomeration process usually entrains certain amount of mother liquor in the agglomerates therefore retains impurities which are supposed to be purged by the crystallization (see K. Funakoshi, H. Takiyama, and M. Matsuoka, “Agglomeration Kinetics and Product Purity of Sodium Chloride Crystals in Batch Crystallization”, Journal of Chemical Engineering of Japan, Vol. 33, No.2, pp267-272, 2000, hereby incorporated by reference), and hence, lower purity of the material generated from the batches described above was observed. The manipulation of supersaturation was consequently not pursued further. However, invaluable information was obtained from the crystallization process-that for this API, nucleation is the rate limiting step for crystallization. This is revealed by two facts:

(1) the formation of agglomerates—typically when nucleation is the bottleneck.

(2) observation of the crystallization process—after seeds are added in step 4 (Table 1), it took more than one hour for the reaction mixture to become a nice and white slurry, much slower than a regular compound where the crystallization usually takes place within 20 minutes with seeding.

Moreover, the manipulation of supersaturation can still quite likely be used in the crystallization of other compounds where the nucleation is fast

To enhance nucleation and preclude growth in the API crystallization, nucleation sites were introduced manually by excessive seeding. Although the current process does involves seeding, the seed loading (“as is” drug at 0.1-0.2% by weight) was not sufficient to effectively relieve supersaturation as well as to maintain the imbalance between nucleation and growth rate. Thus agglomerates or large size elementary crystals with poor compactability are formed. By increasing the seed load the extent of nucleation was significantly improved.

The introduction of more nucleation centers was achieved in a number of ways

1. Increased Seed Loading

On 100 Kg scale using “as is” material at 1.5% seed loading the compactability of the powder blend comprised of 80% bulk drug and 20% excipient doubled from a representative 1.4-1.7 kPa/Mpa (with 0.1% seed loading) to 2.8-3.4 kPa/Mpa. As another example (on 50 g scale) crystallization seeded with 5% large agglomerates the powder blend compactability rose to 3.65 kPa/MPa.

2. Reduction of Seed Particle Size

For the same amount of seed loading (by weight), smaller seeds evidently represent more nucleation centers. Several size reduction strategies were evaluated. The mean particle size of the seeds generated by various comminution methods decreased in the following order: AirJet-milled seeds>seeds crystallized from a ground seeded batch>ball-milled seeds>ground seeds.

After recrystallizing 50-g samples using 1% milled seed. The product compactabilities increased in the following reverse order (i.e. smaller seeds produce API with improved compaction): AirJet-milled seeds (4.2 kPa/MPa)<seeds crystallized from a ground seeded batch (5.3 kPa/MPa)<ball-milled seeds (5.9 Kpa/MPa)<ground seeds (10.5 kPa/MPa).

3. Combination of Seed Load and Size

Examples of 50-g samples are:

i) 1.5% ball-milled seeds—7.0 kPa/MPa

ii) 4% ground seeds—14.4 kPa/MPa—almost a 10-fold improvement over material generated by the current process

iii) 5% ground seeds—12.6 kPa/MPa

In addition to the above nucleation-enhancement strategies, it was further demonstrated in a series of studies that sonication helps induce secondary nucleation, hence improves product compactability even further. API crystallized with 1% ground seeds, without and with sonication show compactabilities of 10.5 kPa/MPa and 12.3 kPa/MPa, respectively.

In order to evaluate the compressibility and compactability of all API lots generated by modifying the crystallization process, a blend of 80% API, 19.5% microcrystalline cellulose and 0.5% magnesium stearate was prepared by mixing in a tumble mixer for 5 minutes. Each mixture was then compressed on an Instron (Universal Stress-Strain Analyzer) using a 0.5 inch diameter tooling (upper and lower punches and die) at a speed of 100 mm/min at compression forces of 5, 10, 15, 20 and 25 kN each for a replicate of three tablets. The tablet dimensions were measured using a digital Vernier calliper and the strength of the tablets were determined using an Erweka hardness tester. The volume of the tablet can be calculated from the tablet dimensions normalized for the true density of the mixture being compressed. The compressibility curves are generated by plotting the solid fraction of the tablet generated at each compression pressure versus the respective compression pressure. The area under such a curve represents the extent of volume reduction. The force required to break the tablets is normalized for the area of the tablet to obtain the tensile strength value. Slopes for profiles of tensile strength versus the compression pressure represent the compactability of the material while the area under the curve of tensile strength versus the solid fraction of the tablets represents the extent of compaction or toughness of the material.

In order to characterize the deformation mechanism of the API, Hiestand's tablet indices (see, E. N. Hiestand and D. P. Smith, Powder Technology, 38, pp 145-159 (1984) hereby incorporated by reference) were evaluated. The identical procedure as developed by E. N. Hiestand, at the Pharmacia and Upjohn company was adapted for evaluating the deformation properties of the API. In brief, square shaped compacts (1.97 cm²) were prepared using a tri-axial decompression Loomis Engineering press. This tri-axial press facilitates compression pressure relief in three dimensions as opposed to two as in the uni-axial press. Hence, it minimizes the shear stresses generated at the compact edges that can lead to false information about the tensile strength of the compacts. Through tri-axial decompression it is possible to produce virtually flawless compacts. The API was compressed with the procedure describe above to produce compacts having a relative density or solid fraction of 0.85. The compacts were then subjected to tensile strength testing on an Instron stress-strain analyzer at a cross head speed of about 0.8 mm/min. This speed allowed the time constant between the peak stress and 1/e times the peak stress to be a constant of 10 seconds. The peak stress required to initiate fracture in the compact in the plane normal to those of the platens of the Instron is used to calculate the tensile strength as shown below:

$$σ = \frac{2 F}{l b}$$

where, σ is the tensile strength calculated and F is the force required to initiate crack propagation in the compact and l and b are the length and breadth of the compact, respectively. MMPI lot# 1 (also known as lot# N0055B) that was prepared with 0.2% w/w seeds during the crystallization process showed tensile strength values of 90.46 N/cm²±5.33 N/cm² for square compacts prepared at a solid fraction of 0.85. On optimizing the crystallization conditions (1.5% w/w seeds of small size) the lot# 2 (also known as lot# R0082) showed tensile strength values of 181.90 N/cm²±9.16 N/cm² for square compacts prepared at a solid fraction of 0.85. Clearly, there is a two fold increase in the tensile strengths for API lots manufactured with the optimized crystallized conditions.

Similarly, the tensile strength is determined for square compacts that are prepared with a magnified flaw using the tri-axial decompression press and a upper punch having a 1 mm diameter pin spring loaded on its surface. This pin facilitates the introduction of a 1 mm diameter hole in the center of the compact. The tensile strength values of the compacts with and without a hole are used to evaluate the brittle fracture index (BFI) of the material as shown below:

$$BFI = [ \frac{{σ}_{T}}{{σ}_{{T}_{0}}} - 1 ] \div 2$$

Where, σ_{T} is the the tensile strength of the square compacts without a hole in the center and σ_{To} is the tensile strength of the square compacts with a 1 mm hole in the center that acts as a stress concentrator. The BFI values of the API, Lot# 1 were found to be 0.14±0.03. Similarly, the BFI values of the API, Lot# 2 were found to be 0.20±0.02. The API shows a brittle fracture index that is on the lower side of the entire (BFI) scale, that ranges from 0 to 1. A value of 0 indicates that the material has very little propensity to show brittle fracture under stress due to predominantly plastic deformation that accommodates the surface stress induced due to the flaw. On the other hand, a BFI value of 1 indicates that the material is unable to accommodate the stress concentration in the center and the flaw in the compact propagates crack growth through the rest of the compact. Hence, it can be concluded that the API shows very little tendency for brittle fracture as its deformation mechanism.

The square compacts (without a hole) are then subjected to a dynamic indentation hardness evaluation using a pendulum impact apparatus as described in Tablet Indices¹¹. The velocity at which the pendulum sphere impacts the compact as well as the speed with which the pendulum sphere is rebound from the compact is recorded. The indentation made on the compact surface by the procedure described above is measured with a surface analyzer that facilitates computation of the chordal radius of the indentation. These measurements are then used to calculate the dynamic indentation hardness of the material using the equation described below:

$$H = \frac{4 {mgrh}_{r}}{π {a}^{4}} ( \frac{{h}_{i}}{{h}_{r}} - \frac{3}{8} )$$

where, m and r are the mass and radius of the indenting sphere, respectively and hᵢ and hᵣ are the inbound and rebound heights, respectively and a is the chordal radius of the indentation created on the compact surface. G is acceleration due to gravity. The dynamic indentation hardness value for the APL Lot # 1, was found to be 35.8 MN/m²±6.2 MN/m². This value is much lower than that of the standard compressible filler, Avicel PH 102 that has a hardness of 352 MN/m². This indicates that MMPI is a very ductile material. The hardness value for Lot # 2 was 52.9 MN/m²±8.2 MN/m². The increase in hardness of the material from the optimized crystallization process is not significant enough to change the conclusion drawn earlier about its ductility.

The Bonding Index of the material can be calculated from the tensile strength measurements as well as the dynamic indentation hardness measurements described above using the equation shown below:

$$BI = \frac{σ}{H}$$

The bonding index of the API was found to be 0.025±0.001. The highest bonding index value observed today is that of microcrystalline cellulose Avicel PH 101 which is 0.04. The bonding index of Lot # 2 was 0.034±0.001. This indicates that the API is a predominantly ductile material.

This example resulted in the formation of a tablet having a very high API load (80% W/W). The final composition of the tablet IS depicted in Table 2.

TABLE 2

| Ingredient | Amount per Tablet |
|---|---|
| API(N-[(2S)-2-Mercapto-1-oxo-4-(3,4,4- | 600.000 mg |
| trimethyl-2,5-dioxo-1-imidazolidinyl)butyl]-L- | |
| leucyl-N,3-dimethyl-L-valinamide) | |
| Microcrystalline cellulose | 97.500 mg |
| Sodium starch glycolate | 37.500 mg |
| Silicon dioxide | 9.375 mg |
| Magnesium stearate | 5.625 mg |
| Total | 750.000 mg |

What is claimed is:

1. A tablet comprising a high active ingredient content wherein said active ingredient is of the general formula (I): 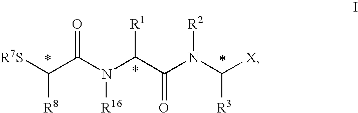
where R¹ is C₁₋₇ alkyl, C₂₋₆ alkenyl, C₁₋₆ alkyl-aryl, aryl, C₁₋₆ alkyl-heteroaryl, heteroaryl or
C₁₋₆ alkyl-AR⁹ group where A is O, NR⁹ or S(O)ₘ where m=0-2, and R⁹ is H, C₁₋₄ alkyl, aryl, heteroaryl, C₁₋₄ alkyl-aryl or C₁₋₄ alkyl-heteroaryl; if A=NR⁹ the groups R⁹ may be the same or different,
R² is hydrogen or a C₁₋₆ alkyl group;
R³ is a R⁶ group where Alk is a C₁₋₆ alkyl or C₂₋₆ alkenyl group and n is zero or 1;
X is heteroaryl or a group CONR⁴, R⁵ where R⁴ is hydrogen or an C₁₋₆ alkyl, aryl, heteroaryl, C₁₋₆ alkyl-heteroaryl, cyclo(C₃₋₆)alkyl, C₁₋₆ alkyl-cyclo(C₃₋₆)alkyl, heterocyclo(C₄₋₆)alkyl or C₁₋₆ alkyl-heterocyclo(C₄₋₆)alkyl group and R⁵ is hydrogen or C₁₋₆ alkyl; NR⁴R⁵ may also form a ring;
R⁷ is hydrogen or the group R¹⁰CO where R¹⁰ is C₁₋₄ alkyl, (C₁₋₄ alkyl)aryl, (C₁₋₆ alkyl)heteroaryl, cyclo(C₃₋₆)alkyl, cyclo(C₃₋₆)alkyl-C₁₋₄ alkyl, C₂₋₆ alkenyl, C₂₋₆ alkenylaryl, aryl or heteroaryl;
R⁸ and R¹⁶ are the same or different and are each C₁₋₄ alkyl R¹¹, R¹⁶ may also be H;
R⁶ represents AR⁹ or cyclo(C₃₋₆)alkyl, cyclo(C₃₋₆)alkenyl, C₁₋₆ alkyl, C₁₋₆ alkoxyaryl, benzyloxyaryl, aryl, heteroaryl, (C₁₋₃ alkyl)heteroaryl, (C₁₋₃ alkyl)aryl, C₁₋₆ alkyl-COOR⁹, C₁₋₆ alkyl-NHR¹⁰, CONHR¹⁰, NHCO₂R¹⁰, NHSO₂R¹⁰, NHCOR¹⁰, amidine or guanidine;
R¹¹ is COR¹³, NHCOR¹³ or any of the groups 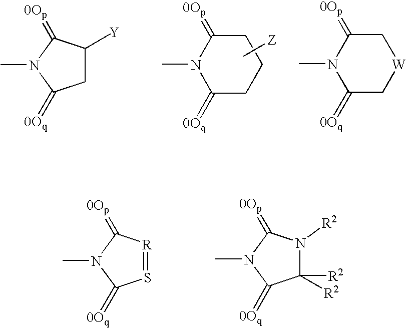
where p and q are each 0 or 1 and are the same or different but when p=q=1, Y cannot be H;
R and S are each CH or N and are the same or different;
W is O, S(O)ₘ where m=0, 1 or 2 or NR¹²;
Y and Z are each H or C₀₋₄ alkylR¹⁴ wherein R¹⁴ is NHR², N(R²)₂ (where each R² may be the same or different), COOR², CONHR², NHCO²R² (where R² is not H), NHSO₂R² (where R² is not H) or NHCOR²; Z may be attached to any position on the ring;
R¹² is hydrogen, C₁₋₄ alkyl, COR⁹, CO₂R⁹ (where R⁹ is not H), CONHR⁹, or SO₂R⁹ (where R⁹ is not H);
R¹³ is (C₁₋₄ alkyl)R¹⁵;
R¹⁵ is N(R²)₂ (where each R⁹ may be the same or different), CO₂R⁹, CONHR⁹, CON(R⁹)₂ (where each R₉ may be the same or different) or SO₂R⁹ (where R⁹ is not H), phthalimido or the groups 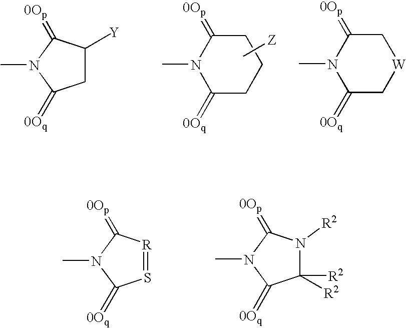
as defined above;
and the salts, solvates and hydrates thereof.

2. The tablet of claim 1 wherein said active ingredient content is greater than 35% of the composition.

3. The tablet of claim 1 wherein said active ingredient content is in the range of about 50% to 90%.

4. The tablet of claim 1 wherein said active ingredient is a compound of formula L wherein X is CONR⁴R⁵; R⁴ is H, alkyl or aryl; R⁶ is not amidine or guanidine; R¹¹ is not NHCOR¹³ or the last of the given groups; R¹⁵ is not N(R²)₂ or the last of the given groups; and R¹⁶ is H.

5. The tablet of claim 1 wherein said active ingredient is a compound of formula I selected from the group consisting of
[(2S)-Sulfanyl-5-[(N,N-dimethylamino)acetyl]aminopentanoyl-L-leucyl-L-tert-leucine N-methylamide;
[(2S)-Sulfanyl-5-[(N-methylamino)acetyl]aminopentnoyl-L-leucyl-L-tert-leucine N-methylamide;
[(2S)-Acetylthio)-4(1,5,5-trimethylhydantoinyl)butanoyl]-L-Leucyl-L-tert-leucine N-methylamide;
[(2S)-Acetylthio)-4(1,5,5-trimethylhydantoinyl)butanoyl]-L-(S-methyl)cysteinyl-L-tert-leucine N-methylamide;
[(2S)-Acetylthio)-4(1,5,5-timethylhydantoinyl)butanoyl]-L-norvalinyl-L-tert-leucine N-methylamide;
N-[2-Sulfanyl-4-(1,5,5-trimethylhydantoinyl)butanoyl]-L-leucyl-L-tert-leucine N-methylamide;
N-[2-Sulfanyl-4-(1,5,5-trimethylhydantoinyl)butanoyl]-L-(S-methyl)cysteinyl-L-tert-leucine N-methylamide; and
N-[2-Sulfanyl-4-(1,5,5-trimethylhydantoinyl)butanoyl]-L-norvalinyl-L-tert-leucine N-methylamide.

6. The tablet of claim 1 wherein said active ingredient is a pharmaceutically active compound of formula I, and the tablet further comprises a pharmaceutically-acceptable diluent or carrier.

7. A pharmaceutical composition comprising at least 35% of an active ingredient having the structure 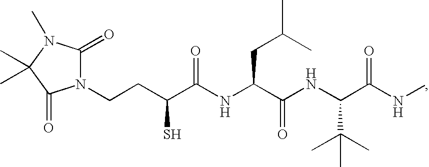
its enantiomers, diastereomers, pharmaceutically acceptable salts, hydrates, prodrugs and solvates thereof.

8. The composition according to claim 7 further comprising at least one excipient.

9. The composition according to claim 7 wherein said active ingredient comprises at least 50% of the composition.

10. The composition according to claim 7 wherein said active ingredient comprises at least 60% of the composition.

11. The composition according to claim 7 wherein said active ingredient comprises at least 70% of the composition.

12. The composition according to claim 7 wherein said active ingredient comprises at least 80% of the composition.

13. The composition according to claim 8 wherein said excipient is selected from the
group consisting of microcrystalline cellulose, sodium starch glycolate, silicon dioxide and magnesium stearate.

14. The composition according to claim 13 wherein said active ingredient is about 50 to 90% of the composition.

15. The composition according to claim 7 further comprising microcrystalline cellulose, sodium starch glycolate, silicon dioxide and magnesium stearate.

16. The composition according to claim 15 wherein said active ingredient is about 70 to 90% of the composition.

17. The composition according to claim 15 wherein said active ingredient is about 80% of the composition; said microcrystalline cellulose is about 13% of the composition; said sodium starch glycolate is about 5% of the composition; said silicon dioxide is about 1.25%; and said magnesium stearate is about 0.75%.

18. The composition according to claim 7 wherein said pharmaceutical composition is in a solid dosage form.

19. The composition according to claim 7 wherein said pharmaceutical composition is a tablet.

20. The composition according to claim 7 wherein said pharmaceutical composition is an oral tablet.
